(19) United States
(12) Patent Application Publication
(10) Pub. No.: US 2007/0111219 A1
(43) Pub. Date: May 17, 2007

(54) Label integrity verification of chemical array data

(76) Inventor: James M. Minor, Cupertino, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/283,453

(22) Filed: Nov. 17, 2005

(51) Int. Cl. C12Q 1/68; G06F 19/00

(52) U.S. Cl. 435/6; 702/20

(57) ABSTRACT

Methods, systems and computer readable media for checking label integrity of labeled biopolymers in a sample assayed by chemical array analysis. A sample is divided into equal aliquots. At least first and second labels are incorporated into biopolymers contained in first and second aliquots of the equal aliquots, respectively. The labels are added to the aliquots in amounts expected to incorporate into the biopolymers of the respective aliquots to produce signals of proportional quantity when read from probes on a chemical array designed to couple with biopolymers of the aliquots. The aliquots are then combined into a single, multi-labeled sample having at least first-labeled biopolymers and second-labeled biopolymers. The multi-labeled sample is hybridized with probes on a chemical array. Signal values are read from the probes on the chemical array bound to labeled biopolymers from the multi-labeled sample. Comparisons are made between signal values from probes bound to biopolymer having the first label incorporated therein (first-labeled signal values) and signal values from the same probes bound to biopolymers having the second label incorporated therein (second-labeled signal values), respectively, from which it is determined that label integrity is of acceptable quality if divergence between the first-labeled signal values and the second-labeled signal values is less than a predetermined threshold value.

### BACKGROUND OF THE INVENTION

Researchers use experimental data obtained from arrays and other similar research test equipment to cure diseases, develop medical treatments, understand biological phenomena, and perform other tasks relating to the analysis of such data. However, the conversion of useful results from this raw data is restricted by physical limitations of, e.g., the nature of the tests and the testing equipment.

All biological measurement systems leave their fingerprint on the data they measure, distorting the content of the data, and thereby influencing the results of the desired analysis. For example, systematic biases can distort array analysis results and thus conceal important biological effects sought by the researchers. Biased data can cause a variety of analysis problems, including signal compression, aberrant graphs, and significant distortions in estimates of differential expression.

Gradient effects or patterns are those in which there is a pattern of expression signal intensity which corresponds with specific physical locations and/or sequence properties within a chemical array and which are characterized by a smooth change in the expression values from one end of the array to another and/or across sequence properties of probes. This can be caused by variations in array design, manufacturing, dye-bias, probe affinity and/or hybridization procedures.

In dual-channel systems, it is well known that the two dyes used to evaluate the binding of target molecules to probes on an array do not always perform equally efficiently, for equivalent target concentrations, uniformly across the whole array. This is sometimes referred to as dye-related, signal correlation bias. For example, for dual-channel systems in which probes have been labeled using cyanine3 (Cy3)- and cyanine5 (Cy5)-dyes, the red channel (detecting Cy5 labeling) often demonstrates higher signal intensity than the green channel at higher target abundances. Even when comparing results from two single-channel experiments, there may be differences in dye performances, even when the same dye is used, such as when different experimental conditions, either intended or unintended, occur when running each of the experiments.

Also, the label intensity may not follow an ideal performance curve over the range of analyte concentration. For example, for drug discovery experiments, label intensity may not follow the ideal dose-response curve over the range of the analyte (e.g., mRNA) concentration being used as a marker of drug efficacy. For example, red dye (e.g., Cy5) tends to amplify brightness in an accelerated manner with respect to an increase in concentration, at high concentrations beyond the typical sigmoidal profile.

The degree the intensity of dye signals fail to report the concentration of target being measured is not easily quantified, and therefore difficult to address.

Dye-swap normalization experiments are sometimes run in which a first set of experiments assigns the red dye label to a first set of probes and the green dye label to a second set of probes. A second set of experiments is run against the same target solution, but in which the green dye label is assigned to the first set of probes and the red dye label is assigned to the second set of probes. By comparing the output of the first set with that of the second set, the bias attributable to the effects of the red versus green dye can be measured. However, this is a time consuming process and significantly increases the cost of experimentation, as twice the amount of arrays, reagents, target and processing are required.

In addition to fluorescent labels, other types of labels, such as radioactive labels, phosphorescent labels, visible light labels, ultraviolet labels, and others, are also susceptible to causing signal correlation bias.

Also, results that appear to have labeling bias may be due to other technical errors. For example, for a single channel system, the system may be erroneously reporting probe signals, even though the results appear to be the cause of dye bias. Since there is only one channel, and no control channel, it is not possible to distinguish between the systematic reader error and dye bias, in this instance.

Thus there remains a need for improved systems and methods for normalizing biological data to address dye-related, signal correlation bias and other types of labeling bias as data is read from arrays.

### SUMMARY OF THE INVENTION

Methods, systems and computer readable media are provided for checking label integrity of labeled biopolymers in a sample assayed by chemical array analysis. A sample is divided into equal aliquots, and at least first and second labels are incorporated into biopolymers in first and second aliquots of the equal aliquots. The labels are added to the aliquots in amounts expected to incorporate into the biopolymers of the respective aliquots to produce signals of proportional value when read from probes on a chemical array designed to bind to biopolymers in the aliquots. The aliquots each having biopolymers with a distinguishable incorporated label (e.g., a spectrally distinguishable label) are then combined to provide a multi-labeled sample, and the multi-labeled sample is hybridized with probes on a chemical array. Signal values are read from the probes on the chemical array bound to labeled biopolymers from the multi-labeled sample. Signal values from probes bound to biopolymers having the first label incorporated therein (“first-labeled signal values”) are compared with signal values from the same probes bound to biopolymers having the second label incorporated therein (“second-labeled signal values”), respectively. Label integrity is determined to be of acceptable quality if divergence between the first-labeled signal values and the second-labeled signal values is less than a predetermined threshold value.

In another embodiment, a chemical array is provided that has had a multi-labeled sample, preparing from labeling equal aliquots of a sample with different labels and then combining the aliquots to provide the multi-labeled sample, contacted thereto so that multi-labeled biopolymers from the same have hybridized with probes on the chemical array. Methods, systems and computer readable media are provided for reading signal values from a probe on the chemical array bound to a set of biopolymer sequences labeled with said at least first and second labels; comparing first-labeled signal values from the probe bound to biopolymer having the first label incorporated therein with second-labeled signal values from the probe bound to biopolymer having the second label incorporated therein; repeating said reading signal values and said comparing first-labeled signal values with second-labeled signal values for at least one additional probe on the chemical microarray bound to a set of different biopolymer sequences labeled with said at least first and second labels; and determining that label integrity is of acceptable quality if divergence between the first-labeled signal values read from the probes and the second-labeled signal values read from the same probes is less than a predetermined threshold value.

These and other advantages and features of the invention will become apparent to those persons skilled in the art upon reading the details of the methods, systems kits and computer readable media as more fully described below.

### BRIEF DESCRIPTION OF THE DRAWINGS

FIG. 1 is a schematic representation of a chemical array.

FIG. 2 is an enlarged view of a portion of the array shown in FIG. 1.

FIG. 3 shows a flowchart of events that may be carried out in processing a sample with multiple different labels.

FIG. 4 is a graphical representation of the number of features provided on the arrays for each of samples in an example described herein.

FIG. 5 shows a plot of the distribution of log ratio values for the signals obtained from scanning arrays in an example experiment described herein.

FIGS. 6A-6C show plots of inter-array coefficient of variation (CV) values calculated for background-subtracted, dye-normalized signals read from arrays in an example experiment described herein.

FIGS. 7A-7C show plots of inter-array coefficient of variation (CV) values (relative noise) similar to FIGS. 6A-6C, except that the signals used for calculations to generate FIGS. 7A-7C were background subtracted, but not dye-normalized.

FIG. 7D shows a plot of inter-array coefficient of variation (CV) values (relative noise) corresponding to the plot of FIG. 7C, except in this case, the signals have been weighted.

FIG. 8 illustrates a typical computer system in accordance with an embodiment of the present invention.

### DETAILED DESCRIPTION OF THE INVENTION

Before the present systems, methods, kits and computer readable media are described, it is to be understood that this invention is not limited to particular methods, method steps, algorithms, software or hardware described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a channel” includes a plurality of such channels and reference to “the array” includes reference to one or more arrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

In the present application, unless a contrary intention appears, the following terms refer to the indicated characteristics.

A “biopolymer” is a polymer of one or more types of repeating units.

Biopolymers are typically found in biological systems and particularly include polysaccharides (such as carbohydrates), and peptides (which term is used to include polypeptides and proteins) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another.

A “nucleotide” refers to a sub-unit of a nucleic acid and has a phosphate group, a 5-carbon sugar and a nitrogen containing base, as well as functional analogs (whether synthetic or naturally occurring) of such sub-units which in the polymer form (as a polynucleotide) can hybridize with naturally occurring polynucleotides in a sequence-specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

“Technical factors” refer to all patterns in the signal data that are not representative of the biological information in the target sample, but are rather caused by technical sources, such as hybridization bubbles (caused by uneven distribution of the sample to all probes during mixing by a bubbler), temperature gradients, sequence-composition gradients, writer/pen anomalies causing uneven patterns in the amounts deposited across the array, label kit biases, dye differences, bulk chemical solution effects, flow-cell dynamics, wash deposits, auto-fluorescence, oxidation gradients, and the like.

“Incorporation” of a label, into biopolymers or nucleotides, for example, refers to any known technique for labeling a biopolymer or nucleotide, including, but not limited to primer extension using labeled nucleotides and/or labeled primers, labeling during an amplification procedure, chemical conjugation, labeling by binding a labeled moiety that binds to the biopolymer, etc.

“Label integrity”, as used herein refers to a property of labels incorporated into biopolymers wherein signals that are read from the label-incorporated biopolymers can be consistently and stably reproduced across multiple experiments. Also, different labels vary proportionally over a range of signals, so that they can be reliably compared with one another, as measuring the same signal levels for the same sample, or correct ratios between different samples. Labels that lack label integrity are considered unstable, and this leads to amplified array noise and the inability to accurately compare signals from the same biopolymers labeled with different labels. Stability with respect to time (e.g., “shelf life”) is also a desirable property for maintaining label integrity.

When one item is indicated as being “remote” from another, this is referenced that the two items are not at the same physical location, e.g., the items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

A “chemical array”, “array”, “microarray” or “bioarray” unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other).

An “array layout” refers to one or more characteristics of the features, such as feature positioning on the substrate, one or more feature dimensions, and an indication of a moiety at a given location.

“Hybridizing” and “binding”, with respect to polynucleotides, are used interchangeably.

A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice).

A “subarray” or “subgrid” is a subset of an array. Typically, a number of subgrids are laid out on a single slide and are separated by a greater spacing than the spacing that separates features or spots or dots.

Any given substrate (e.g., slide) may carry one, two, four or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features).

Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible; for example, some manufacturers are currently working on flexible substrates), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. Nos. 6,242,266; 6,232,072; 6,180,351; 6,171,797; and 6,323,043, and in U.S. patent application Ser. No. 09/302,898 filed Apr. 30, 1999 by Caren et al., and the references cited therein. As already mentioned, these references are incorporated herein, in their entireties, by reference thereto. Other drop deposition methods can be used for fabrication, as previously described herein. Also, instead of drop deposition methods, photolithographic array fabrication methods may be used. Interfeature areas need not be present particularly when the arrays are made by photolithographic methods.

Following receipt by a user of an array made by an array manufacturer, it will typically be exposed to a sample (for example, a fluorescently labeled polynucleotide or protein containing sample) and the array then read. Reading of the array may be accomplished by illuminating the array and reading the location and intensity of resulting fluorescence at multiple regions on each feature of the array. For example, a scanner may be used for this purpose which is similar to the AGILENT MICROARRAY SCANNER manufactured by Agilent Technologies, Palo Alto, Calif. Other suitable apparatus and methods are described in U.S. Pat. Nos. 6,406,849; 6,371,370; and 6,756,202; and in U.S. Patent Publication No. 2003/0160183 titled “Reading Dry Chemical Arrays Through The Substrate” by Dorsel et al. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685 and 6,221,583 and elsewhere). A result obtained from the reading followed by a method of the present invention may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

The term “stringent assay conditions” or “stringent conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

As noted above, conventional bioassays use one dye label per signal channel, with no direct onboard way to assure integrity of the label dyes. Examples of widely-used single-channel platforms include GeneChip®, by Affymetrix (http://www.affymetrix.com/products/arrays/index.affx) and the CodeLink System from GEHealthcare (http://www.affymetrix.com/products/arrays/index.affx). A gradient pattern that results from reading such an array does not necessarily imply a dye-biasing error, but could be due to other production factors during production of the array and/or hybridization conditions, as noted above. Further, with single-channel systems, since there is only one channel being analyzed, it is not possible to run dye-swap experiments, as there is typically only one set of probes and one dye used.

The present invention provides solutions that include onboard verification of labeling, even for single-channel systems. Multiple labels may be incorporated into one sample, such that the probes on an array read by a single channel of a system will get information from multiple labels. For example, for dye-biasing, both red and green dye labels may be incorporated in the same sample containing target nucleic acids and the multi-labeled sample is then exposed to the probes on an array under stringent hybridization conditions. The multiple dye labels may be incorporated separately into equal aliquots of the same sample (e.g., comprising equal concentrations of biopolymers) and then combined to provide the multi-labeled sample, or incorporated all at once into a single aliquot of the sample to produce the multi-labeled sample. The resulting signals read by an array scanner will then reflect the same sample labeled with green dye, as well as with red dye. Thus, a two-channel, or two color scanner may be used to process a single sample in this instance, with one channel of signal measurement.

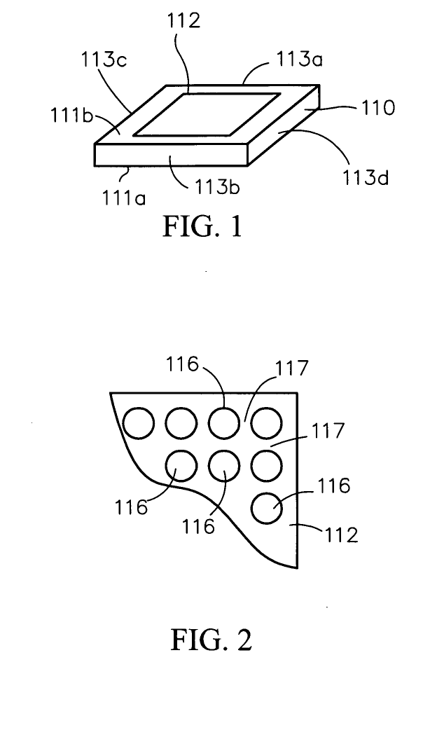

FIGS. 1-2 illustrate an exemplary array, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a surface of the substrate and depending on the use of the array, any or all of the arrays may be the same or different from one another and each may contain multiple spots or features. The one or more arrays 112 usually cover only a portion of the surface 111b, with regions of the surface 111b adjacent the opposed sides 113c, 113d and leading end 113a and trailing end 113b of slide 110, not being covered by any array 112. An opposite surface 111a of the slide 110 typically does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of oligomers, e.g., in the form of polynucleotides, and specifically oligonucleotides. As mentioned above, all of the features 116 may be different, or some or all could be the same. The interfeature areas 117 could be of various sizes and configurations. Each feature carries a predetermined oligomer such as a predetermined polynucleotide (which includes the possibility of mixtures of polynucleotides). It will be understood that there may be a linker molecule (not shown) of any known types between the surface 111b and the first nucleotide.

Substrate 110 may carry on surface 111a, an identification code, e.g., in the form of bar code (not shown) or the like printed on a substrate in the form of a paper label attached by adhesive or any convenient means. The identification code may contain information relating to array 112, where such information may include, but is not limited to, an identification of array 112, i.e., layout information relating to the array(s), etc.

In the case of an array in the context of the present application, the “target” may be referenced as a moiety in a mobile phase (typically fluid), to be detected by “probes” which are bound to the substrate at the various regions.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

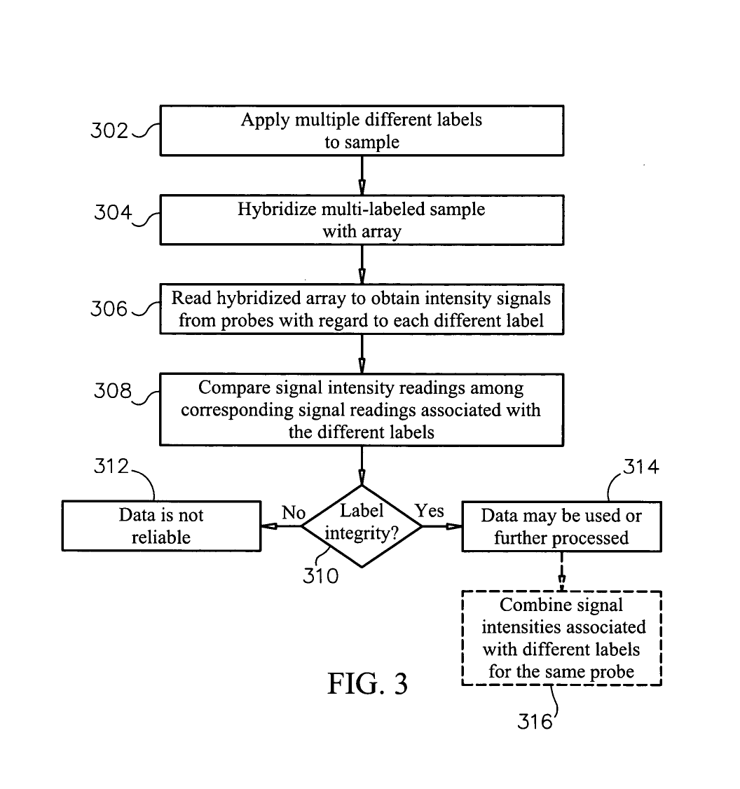

FIG. 3 shows a flowchart of events that may be carried out in processing a sample with multiple different labels. At event 302, multiple different labels are applied to the same sample in proportions, such that each label produces proportional signals on each probe. That is, a single sample containing target nucleic acids may be divided into equal aliquots, one for each different type of label to be incorporated therein. Then, each different type of label is incorporated into a respective aliquot, and the labeled aliquots are mixed together to provide one quantity of multi-labeled sample. Even more reliable is to process a single aliquot of the sample with a solution having a mixture of labels added in known proportional amounts (e.g., such as equal amounts or amounts that will produce equal signals or one signal in a multiple of the other signal in proportion across probes on an array) for incorporation of the multiple labels into the single sample in a single labeling process. Next, at event 304, the multi-labeled sample is hybridized with probes on an array having probes designed to bind with polynucleotides that are expected to be present in the sample. Replicates of probes may be provided on the array. Upon hybridizing the array with the target, multi-labeled sample, each probe is expected to bind with numbers or concentrations of each label to produce proportional signals or scanner counts, as incorporated in the specific polynucleotide that that probe is designed to bind with, since labels were applied to the aliquots of the sample in relative numbers or concentrations calculated to produce proportional signals or scanner counts for the same probe. Ideally, equal signals are produced, but this is not necessary, since a comparison of patterns (e.g., gradients) across the signals received from the probes is what is important, not a comparison of signal magnitudes per se. Conversion methods can be applied when comparing unequal signal magnitudes, as taught in U.S. Pat. No. 6,188,969 and/or in U.S. Patent Publication No. 2005/0143935, both of which are incorporated herein, in their entireties, by reference thereto.

After washing and other typical processing steps, the array is then processed at event 306 to read the array (such as by scanning, or the like) to obtain signals from the probes with regard to each different label, respectively. The signal values associated with each of the different labels for each probe may then be used as a measure of label integrity, i.e., to measure the fidelity of the signals as effected by one label versus the others. Additionally, the signal values associated with each of the different labels may be used to improve quantitation and reproducibility of signal quantitation results, as will be described below. Thus, the techniques described herein describe an onboard diagnostic test of the labels employed, which may be used in experimental arrays for improving quality of results from arrays actually used in running experiments.

Since each label is expected to be incorporated into the sample in proportions designed to produce proportional signal levels on the same probe, each set of signals for each label, respectively, are expected to measure the same biopolymers (e.g., polynucleotides) in equal concentrations for each probe. Thus, a comparison of the signals associated with each label provides a reliable measure of whether the labels are distorting the signal readings, since all other technical factors do not vary (e.g., array to array differences, lot to lot differences, hybridization conditions, array manufacturing conditions, etc., that may typically be causes of gradients and other pattern variations under circumstances comparing two samples from two different arrays or, at least some of these may also be factors when comparing two samples on two channels of the same array).

The signal intensity values associated with the different labels are then compared at event 308 to identify label-induced errors (i.e., errors resulting from a lack of label integrity) in the signal intensities, or to confirm label integrity. One technique for comparison involves calculating (and optionally, plotting) response surfaces for each set of signals (where each set is associated with a different label) against the locations of the probes on the array from which the signals were obtained. Response surfaces may be plotted using any of a number of known techniques. The response surfaces should generally follow the same contour to confirm that label integrity exists, since the other technical factors (e.g., hybridization differences, array production and processing differences, etc., between experiments) are effectively eliminated by processing the same single sample on the same array, with respect to all labels. If a response surface associated with any particular label diverges from the response surfaces associated with the other label or labels, then this is an indication of error induced by one or more of the labels. A divergence threshold may be set that defines acceptable performance. For example, if customers require the median inter-array coefficient of variation percentages (% CV) to be 10% or less, then a volatile, non-persistent ratio gradient associated with % CV>10% is not acceptable.

Thus, for example, if the response surfaces generated from signals associated with labels 2, 3 and 4, respectively generally follow the same contours, but the response surface generated from signals associated with label 1 follows significantly different contours along all or a portion of the response surface, then this is indication that there may be a problem with the label integrity of label 1. When only two labels are used, it may be indeterminate as to whether one or the other label (or both) are lacking in integrity. However, in any of the preceding instances, the result is the same, in that the results of an array experiment would be unreliable or unacceptable for lack of label integrity.

Another technique for comparison includes calculating log ratios of intensity signal pairs, associated with different labels, but the same probe. Signal pair ratios may be calculated for all possible combinations of different pairs of different labels, for each probe. For any given probe, each different label referred to is incorporated in the same target biopolymer (for example, the same target nucleic acid) of the sample which that probe is designed to bind with. In this case, the ratios calculated are not expression ratios or ratios to indicate other signals characterizing the sample (e.g., indicating copy number, as in a CGH assay or transcription factor binding sites, as in a location analysis assay) but rather are ratios of the same signal reading, but where each intensity signal from the pair is associated with a different label (i.e., the same biopolymer sequences bind to a probe, but the sequences have different labels. Assuming that the labels perform equally, the calculated log ratios should have a value of zero. However, there may be some bias between labels. For example, dye bias is known to be possible, such that a red dye associated with the same polynucleotide as a green dye may result in a higher signal intensity reading with regard to the polynucleotide incorporating the red dye relative to the polynucleotide incorporating the green dye. In these instances, the data may be processed to remove label biasing, by any variety of known techniques. However, with or without processing to remove label biasing, the log ratio values should remain fairly consistent across all probes on the array if there is label integrity. That is, even with dye bias being present, the log ratio of signal values associated with two different labels, from a first probe should be the same as the log ratio of signal values associated with those same two different labels from a second probe, if label integrity exists. In other words, the difference between the log ratio of signal values associated with two different labels, from a first probe, and the log ratio of signal values associated with those same two different labels from any other probe on the array should be zero, or within a predetermined threshold value (positive difference less than the threshold value, negative difference greater than the negative of the threshold value), if label integrity exists. Another example is that if other technical factors exist that would cause a gradient in the surface response for signal intensities associated with label 1, then those technical factors will also exist with regard to the signal intensities associated with label 2, so that although the surface response associated with each of labels 1 and 2 will each show a gradient, a response surface generated from the ratios or log ratios of the signal associated with label 1 to the signals associated with label 2 (or vice versa) will not have the gradient, indicating that the gradient in the response surfaces associated with the single labels is induced by technical factors other than the labels themselves.

After comparison of the signal intensity readings associated with the different labels, a determination may be made, based on such comparison, as to whether the fidelity of the signal intensity readings, as impacted by the labels used, is reliable. If it is determined that one or more labels lack integrity, such as by observing significant divergence of response surfaces, or variation in the differences between ratios across the array, then label integrity is determined to be absent at event 310 and the data is considered to be unreliable at event 312. Unstable labeling tends to amplify all differences such as the chemical differences between two different label dyes, for example. On the other hand, if label integrity is found to exist at event 310, then the data (signal intensity readings) may be considered reliable, at least to the extent that the labels used are not distorting the signal intensity readings.

It has been further discovered that the signal intensity readings associated with the different labels may be combined to form a composite or average signal intensity level for a probe, which may be more accurate, reliable and reproducible across experiments than if any single signal intensity level associated with any single label associated with the experiment was used. Such processing may optionally be carried out at event 316. The technique can average out small inconsistencies that may be present with various different types of labels. For example, labels such as dyes may exhibit a small amount of abundance-dependence, such as when dyes are incorporated into RNA according to the number of opportunities present (i.e., the number of nucleic acids that are present and complementary to the labeled nucleic acids). One observation has been that the red dye label Cy5- is incorporated faster than the green dye label Cy3- at higher abundances. By averaging the signals, the effects of abundance dependence of one of the labels is reduced by the values associated with the other labels that are not abundance dependent in that range of signal levels. As a simple example, if label 1 amplifies the signal somewhat at lower abundances and thus provides stronger signals at lower signal levels reflective of lower abundance of the sample on a probe and label 2 does not, then by averaging the signals the amplification is reduced.

As another approach to multiple labeling of a sample, two or more different labels (of any of the types described previously) may be incorporated in the same sample, and the labeled target biopolymers in the sample are contacted to probes on the features 116 of the array. For example, cyanine3—(Cy3) and cyanine5—(Cy5) dye labels may be mixed in amounts determined (e.g., empirically) to produce proportional signals or scan counts for the same probe applied thereto, and then combined with a tissue sample (e.g., spleen, or other tissue sample to be analyzed). Further details about applying multiple labels in this manner can be found in co-pending application Ser. No. (application Ser. No. not yet assigned, Attorney's Docket No. 10051064-1) filed concurrently herewith and titled “Label Integrity Verification of Chemical Array Data”, which is hereby incorporated herein, in its entirety, by reference thereto. The multiple-labeled sample (target sample) may then be contacted to a microarray having probes designed to bind with particular biopolymer (e.g., polynucleotide) sequences expected to be contained in the target sample. The array may contain oversampled probes, i.e., one or more, and up to all of the specifically designed probes may be provided in more than one feature 116 each, so that multiple features are provided to measure the same biopolymer sequence.

An example of the approach where different labels are incorporated into separate equal aliquots of the same sample, then mixed into a single sample and hybridized to probes on an array, follows. Although the specific example is directed to dye labeling, it is again noted here that the principles and methods described herein are equally applicable to other label types. For example, the same sample may be labeled with either Cy3-dye or Cy5-dye and labeled with a radioactive label, as well, or with two radioactive labels (radioactive isomers), biotinylated dyes, or with two different labels of any known types, as long a system or systems are available for reading the signals associated with such labels.

In the following example, two different dye labels were incorporated into separate equal aliquots of the same sample, then mixed into a single sample and hybridized to probes on an array. The example experiment was conducted on self-self arrays in which equivalent proportions of cyanine3—(Cy3) and cyanine5—(Cy5) dye labels were separately incorporated in nucleic acids in equal, but separate quantities of the same sample, and both samples were hybridized, under the same conditions to the same array configured for two channel processing, commonly referred to as “self-self hyb”, under the following conditions:

For a self-self hybridization, 1 μg of Hela or K562 total RNA was amplified and Cy3- and Cy5-labeled using Agilent's Low Input RNA Fluorescent Linear Amplification Kit (5184-3523, Agilent Technologies, Inc., Palo Alto, Calif.) in separate reactions, following protocol described in the user's manual of the kit. Hybridizations were performed using Agilent's Human 1A (V2) Oligo Microarrays (G4110B, Agilent Technologies Inc., Palo Alto, Calif.) and the in-situ Hybridization Plus Kit (5184-3568, Agilent Technologies, Inc., Palo Alto, Calif.). 750 ng of Cy3- and 750 ng of Cy5-labeled cRNA were co-hybridized to each microarray, as described in the microarray user manual (G4140-90030, Agilent Technologies, Inc., Palo Alto, Calif.). Slides were scanned on an Agilent Microarray Scanner (Model G2505B, Agilent Technologies, Inc., Palo Alto, Calif.) and the raw images were processed using Agilent's Feature Extraction (v7.5.1, Agilent Technologies, Inc., Palo Alto, Calif.).

This experiment was closely controlled to provide the same technical factors to both samples on the same array, to validate the usefulness of providing two or more labels to the same sample to monitor label integrity as described herein. Table 1 lists the four Agilent oligo, two-color arrays (self 3, self4, self 7 and self8) that were prepared for the experiment. The arrays self3 and self7 used HeLa_{—}11 as the sample for both red and green dyes in equal proportions, and the arrays self4 and self8 used K562_{—}12 as the sample for both red and green dyes in equal proportions.

TABLE 1

| Array | Barcode | RedSample | GreenSamp | Description |
|---|---|---|---|---|
| self3 | 16011877010 | Cy5 HeLa | Cy3 HeLa | Cy3 HeLa + Cy5 HeL |
| self4 | 16011877010 | Cy5 K562 | Cy3 K562 | Cy3 K562 + Cy5 K562 |
| self7 | 16011877010 | Cy5 HeLa | Cy3 HeLa | Cy3 HeLa + Cy5 HeL |
| self8 | 16011877010 | Cy5 K562 | Cy3 K562 | Cy3 K562 + Cy5 K562 |

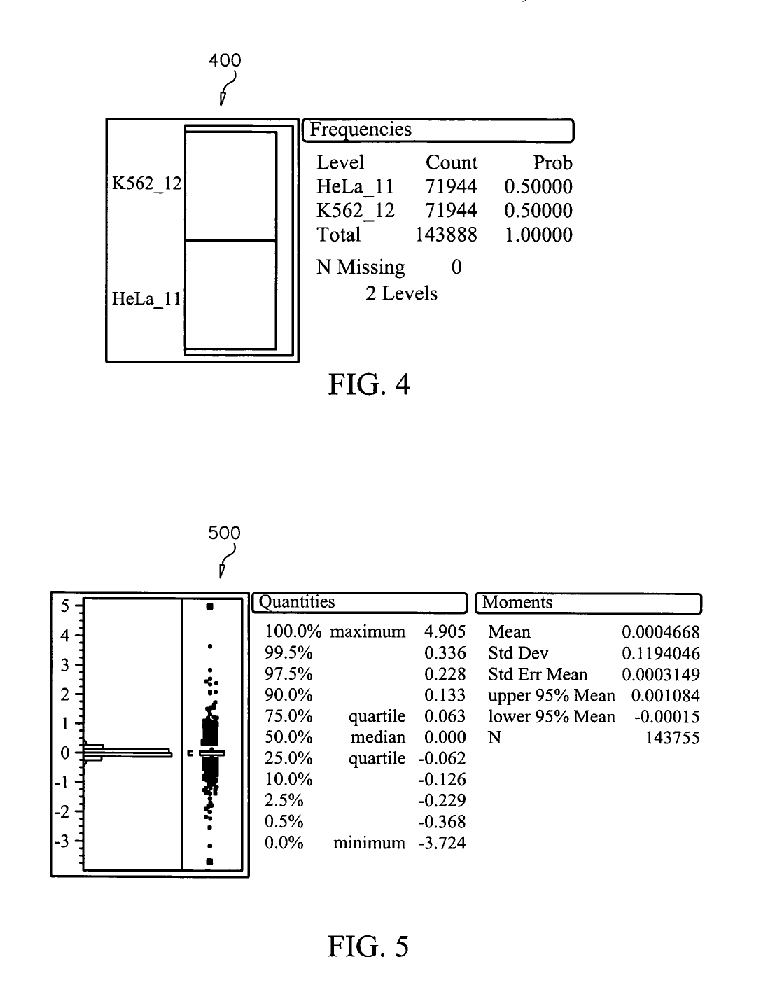

FIG. 4 is a graphical representation of the number of features provided on the arrays for each of samples HeLa_{—}11 and K562_{—}12, as an overall count for arrays self3, self4,self7 and self 8 combined, as well as the numerical totals for each and the total overall. As noted in FIG. 4, there were 71,944 probes designed for the HeLa_{—}11 sample and 71,944 probes designed for the K562_{—}12 sample. As noted above, the signal intensity ratios between red and green labeled signals for the same probe measure the integrity of the dye, rather than expression ratios. More specifically, these ratios measure dye parallelism, where a plot of ratio values from probe to probe should be fairly constant (with the exception of random noise), even if ratio values are not zero.

Upon hybridizing each array with the target samples as indicated above, each probe was ideally expected to bind with equal concentrations Cy3-labeled polynucleotides and Cy5-labeled polynucleotides of the specific polynucleotide that is designed to bind with.

After washing and other typical processing steps, the arrays were scanned with a two-channel Agilent scanner to obtain signals from the probes for both the Cy3-labeled target as well as the Cy5-labeled target on the two channels, respectively. The ratios of the signal values from the two channels for each probe were than analyzed as a measure of dye integrity, i.e., to measure the fidelity of the signals as effected by one dye versus the other.

Since both channels were expected to measure the same biopolymers (e.g., labeled polynucleotides) present in equal concentrations for each probe, a comparison of the signals from each channel with the processing described herein, provides a reliable measure of whether the labels are distorting the signal readings, since all other technical factors do not vary (e.g., such as one or more of: array to array differences, lot to lot differences, hybridization conditions, array manufacturing conditions, etc., factors that may typically cause gradients and other pattern variations when comparing two samples contacted to two different arrays.

It should be further noted here that the present invention is not limited to the use of two different labels with the same sample, as more than two different labels may be applied to perform the functions described herein, and which would be processed similarly. By using a mixture of multiple (two or more) different labels for the same sample, the signal readings associated with each individual label may compared with the signal reading associated with each of the other individual labels, thereby providing a check of integrity of the labels used and hence, fidelity of the signals read. For example, if use of one particular label, for example, a dye, results in signal levels read during processing that when plotted against the positions of the features from which the signals were read, presents an unusual gradient in the surface response plot characterizing the plotted signal levels, as compared to surface response plots for the other labels, then this is evidence that the dye has a lack of integrity across the range of signal levels read. For example, Cy5 label (red) is more susceptible to ozone degradation than Cy3 label (green). Another example is that auto-fluorescence can influence signals from sample labeled with Cy3 dye much more than signals from samples labeled with Cy5 dye. In situations such as these, the signals read from sample labeled with red dye and the signals read from sample labeled with green dye result in a mutually divergent pattern when the signals are plotted with regard to the positions of the features on the array to produce surface response plots, since chemical differences are amplified by unstable conditions.

By providing multiple labels in a manner described with a universal reference (i.e., a reference designed to use for a broad coverage of different gene expression studies, e.g., see http://www.stratagene.com/products/displayProduct.aspx?pid=439), label integrity can be checked by comparison of signals as described, as read from the biopolymers on the universal reference that have been labeled with multiple labels, thus providing an experimenter with assurance that the labels associated with experimentation are not a significant source of error and assay instability.

FIG. 5 shows a plot 500 of the distribution of log ratio values for the signals obtained from scanning all four of the arrays identified in Table 1 above, where each log ratio value is the log ratio of an intensity signal associated with the red dye to the intensity signal associated with green dye, for the same probe/target on the same array. It can be observed that the distribution of the log ratio values shows that the log ratio values are centered around zero, as expected. The associated statistics shown in FIG. 5 indicate that the median ratio value is zero, with 25th and 75^{th} percentile values being within 0.063 of zero, with a tight distribution, indicating a relatively low amount of random noise.

As one approach to analysis of the array data from scanning the arrays identified in Table 1, ANOVA analysis of the signal data obtained from the arrays was performed using JMP*SAS software (http://wwwjmp.com/) to characterized the response surfaces and check for relative dye patterns in the signal intensities, as measured by natural log ratios of dye-normalized, background subtracted signals (LnRatiOrgDNS) for red to green ratios from the probes/targets on the arrays. The ratios are analyzed to look for patterns of divergence caused by differences in performance of the red and green dyes. The analysis performed was standard ANOVA analysis to measure the dye integrity for the arrays noted. Further information regarding ANOVA analysis can be found in co-pending, commonly assigned application Ser. Nos. 11/198,362, filed Aug. 4, 2005 and Ser. No. 11/026,484, filed Dec. 30, 2004. Both application Ser. No. 11/198,362 and application Ser. No. 11/026,484 are hereby incorporated herein, in their entireties, by reference thereto. Table 2 shows summary results for the surface fit and the Analysis of Variance Results as determined by the ANOVA processing.

TABLE 2

| | | Analysis of Variance | | | | |
|---|---|---|---|---|---|---|
| Summary of Fit | | Source | DF | SSQ | Mean Square | F Ratio |
| RSquare | 0.015855 | Model | 23 | 32.4955 | 1.41285 | 100.6756 |
| RSquare Adj | 0.015697 | Error | 143731 | 2017.0715 | 0.01403 | Prob > F |
| RMS Error | 0.118464 | C. Total | 143754 | 2049.5670 | | 0.0000 |
| Mean of Resp | 0.000467 | | | | | |
| Sum Wgts | 143755 | | | | | |

Table 2 reports well-known, established standard statistics for an ANOVA analysis. In the “Summary of Fit” portion of Table 2 above, “RSquare” measures the proportion of the variation around the mean explained by the linear or polynomial model. The remaining variation is attributed to random error. RSquare is 1 if the model fits perfectly. An RSquare value of zero indicates that the fit is no better than a simple mean model. RSquare is the standard regression result of one minus the ratio residual sum of squares, divided by the total sum of squares, about the mean. “RSquare Adj.” adjusts the RSquare value to make it more comparable over models with different numbers of parameters by using the degrees of freedom in its computation. Thus it is a ratio of mean squares instead of sums of squares.

“RMS Error”, or “Root Mean Square Error” estimates the standard deviation of the random error. RMS Error is calculated as the square root of the mean square for Error in the Analysis of Variance table shown in the “Analysis of Variance” portion of Table 2. “Mean of Response” is the sample mean (arithmetic average) of the response variable. This is the predicted response when no model effects are specified. “Sum of Weights”, or “Observations”, indicates the number of observations used to estimate the fit, in this case, the number of rows of data that were inputted.

In the “Analysis of Variance” portion of Table 2 above, “DF” refers to the degrees of freedom for each calculation reported. The Total Error DF is the degrees of freedom figure reported at the “Error” entry of the Analysis of Variance portion of Table 2, and is the difference between the “C. Total” DF value and the “Model” DF value. The Sum of Squares or “SSQ” records an associated sum of squares for each source of error. The Total Error “SSQ” is the sum of square value reported on the “Error” line of the Analysis of Variance portion of Table 2.

“Mean Square” is the sum of squares divided by it associated degrees of freedom, i.e., SSQ/DF. This computation converts the sum of squares to an average (mean square). “F Ratio” is the ratio of mean square for lack of fit to mean square for pure error. The F-Ratio tests the hypothesis that the lack of fit error is zero. F-ratios for statistical tests are the ratios of mean squares. “Prob>F” is the observed significance probability (p-value) of obtaining a greater F-ratio value by chance alone if the specified model fits no better than the overall response mean (i.e., probability of a noise effect). Observed significance probabilities (Prob>F) of 0.05 or less are often considered evidence of a regression effect.

Table 3 shows the parameter estimates that were calculated for performing the ANOVA analysis. The nominal terms inputted were the self-self arrays (ArraySelf3, ArraySelf4 and ArraySelf7) with the array self8 (ArraySelf8) serving as the intercept term, as one of the nominal terms (levels) becomes the designated dependent effect to be left out of the model to avoid singularity problems. This parameter becomes the negative of the sum of all other level parameters and therefore absorbs the singularity. The “Estimate” column lists the parameter (term) estimates of the linear model. The prediction formula is the linear combination of these estimates with the values of their corresponding variables. “Std. Err.” lists the estimates of the standard errors of the parameter estimates. These Std. Err. estimates are used for constructing tests and confidence intervals.

The “t Ratio” column lists the test statistics for the hypothesis that each parameter is zero. The t Ratio is the ratio of the parameter estimate to its standard error. If the hypothesis is true, then this statistic has a Student's t-distribution. Looking for a t Ratio greater than 2 in absolute value is a common rule of thumb for judging significance because it approximates the 0.05 significance level.

The final column labeled “Prob>_{|t|}” lists the observed significance probability calculated from each t Ratio. Prob>_{|t|} is the probability of getting, by chance alone, a t Ratio greater (in absolute value) than the computed value, given a true hypothesis. Often, a value below 0.05 (or sometimes 0.01) is interpreted as evidence that the effect of the parameter considered is significantly different from zero. The different values in this column for the nominal variables ArraySelf3, ArraySelf4 and ArraySelf7 indicate LnRatio shifts due to variation in the amount of response of the red dye relative to the green dye for the same probe/target, over all of the probes on the arrays among the arrays, respectively. ANOVA nominal variables are composed of dummy values which represent shifts as estimated by their parameters. The shifts were considered to be within an acceptable range in this example. An acceptable range may be preset to make this determination. For example, in this example, the range was preset for a determination that a shift was in an acceptable range if the p-value was less than 0.05, which is a typical threshold setting for significance.

The second grouping of terms in Table 3 (i.e., Col&RS, (Row-103.983)* (Row-103.983), (Row-103.983)* (Col-215.455), and (Col-215.455)* (Col-215.455)), are scaled or covariate terms, minus their average value (to improve numerical and statistical properties), and provide the statistical results that characterize the global, persistent (array independent pattern) effects, to the second order, of the row and column positions of the probes on the arrays with respect to all four of the arrays (ArraySelf3, ArraySelf4, ArraySelf7 and ArraySelf8) considered together, upon the outcome of the signal levels (natural log ratios of dye-normalized, background subtracted signals, in this example). Note that the numerical values “103.983” and “215.455” are the average row and column positions on an x-y grid, as measured on the array by the analysis software, and that these values are subtracted from each row and column position, respectively, to center the data for performance of the analysis, thereby reducing effect correlations. Specifically, in this example, Col&RS characterizes the effect of the column positions, (Row-103.983)* (Row-1 03.983) characterizes the second order effect of row positions, or row-row interaction (i.e., row²), (Row-103.983)* (Col-215.455) characterizes the effect of row and column interaction, and (Col-215.455)* (Col-215.455) characterizes the second order effect of column positions, or column-column interaction (i.e., column²) Given the extremely low p-values in the last column for these terms, this indicates that persistent gradients apply to all the arrays considered, in the LnRatiOrgDNS data, but that these gradients are very small as indicated by the small parameter estimates for these terms.

The third grouping of terms in Table 3 (i.e., (Row-103.983)*ArraySelf3, (Row-103.983)*ArraySelf4, (Row-103.983)*ArraySelf7, (Col-215.455)*ArraySelf3, (Col-215.455)*ArraySelf4, (Col-215.455)*ArraySelf7, (Row-103.983)*(Row-103.983)*ArraySelf3, (Row-103.983)*(Row-103.983)*ArraySelf4, (Row-103.983)*(Row-103.983)*ArraySelf7, (Row-103.983)*(Col-215.455)*ArraySelf3,

TABLE 3
Parameter Estimates

| Term | Estimate | Std. Err. | t Ratio | Prob>\|t\| |
|---|---|---|---|---|
| Intercept | 0.0232386 | 0.000972 | 23.91 | <.0001 |
| ArraySelf3 | 0.0033311 | 0.001014 | 3.29 | 0.0010 |
| ArraySelf4 | 0.0013103 | 0.001014 | 1.29 | 0.1963 |
| ArraySelf7 | 0.0013831 | 0.001014 | 1.36 | 0.1726 |
| Row&RS | −0.000085 | 0.000005 | −16.09 | <.0001 |
| Col&RS | −0.000018 | 0.000003 | −7.23 | <.0001 |
| (Row-103.983)*(Row-103.983) | 5.4806e−7 | 9.907e−8 | 6.63 | <.0001 |
| (Row-103.983)*(Col-215.455) | 6.8524e−7 | 4.263e−8 | 16.07 | <.0001 |
| (Col-215.455)*(Col-215.455) | −7.786e−7 | 2.271e−8 | −34.28 | <.0001 |
| (Row-103.983)*ArraySelf3 | 0.0000458 | 0.000009 | 5.01 | <.0001 |
| (Row-103.983)*ArraySelf4 | 0.0000496 | 0.000009 | 5.44 | <.0001 |
| (Row-103.983)*ArraySelf7 | −0.000001 | 0.000009 | −0.15 | 0.8841 |
| (Col-215.455)*ArraySelf3 | −0.000019 | 0.000004 | −4.42 | <.0001 |
| (Col-215.455)*ArraySelf4 | −0.000032 | 0.000004 | −7.23 | <.0001 |
| (Col-215.455)*ArraySelf7 | −0.000021 | 0.000004 | −4.83 | <.0001 |
| (Row-103.983)*(Row-103.983)*ArraySelf3 | 1.9264e−7 | 1.716e−7 | 1.12 | 0.2616 |
| (Row-103.983)*(Row-103.983)*ArraySelf4 | −0.000001 | 1.716e−7 | −6.14 | <.0001 |
| (Row-103.983)*(Row-103.983)*ArraySelf7 | 5.55393−7 | 1.716e−7 | 3.24 | 0.0012 |
| (Row-103.983)*(Col-215.455)*ArraySelf3 | −4.804e−8 | 7.383e−8 | −0.65 | 0.5152 |
| (Row-103.983)*(Col-215.455)*ArraySelf4 | −3.04e−8 | 7.385e−8 | −0.41 | 0.6806 |
| (Row-103.983)*(Col-215.455)*ArraySelf7 | 2.1317e−8 | 7.384e−8 | 0.29 | 0.7728 |
| (Col-215.455)*(Col-215.455)*ArraySelf3 | −6.149e−8 | 3.934e−8 | −1.56 | 0.1180 |
| (Col-215.455)*(Col-215.455)*ArraySelf4 | 1.0122e−8 | 3.934e−8 | 2.57 | 0.0101 |
| (Col-215.455)*(Col-215.455)*ArraySelf7 | −8.415e−8 | 3.934e−8 | −2.14 | 0.0324 |

(Row-103.983)*(Col-215.455)*ArraySelf4, (Row-103.983)*(Col-215.455)*ArraySelf7, (Col-215.455)*(Col-215.455)*ArraySelf3, (Col-215.455)*(Col-215.455)*ArraySelf4, and (Col-215.455)*(Col-215.455)*ArraySelf7) are scaled or covariate terms, per array, that characterize the changes in LnRatiOrgDNS values for each array, on a per array basis, respectively, as effected by row and column positions of the probes/targets on the arrays. These parameters indicate the shift in the persistent parameters for each array for all gradient effects.

Specifically, “(Row-103.983)*ArraySelf3” characterizes the row effect shift upon any gradient that may be observed in array self3. (Row-103.983)*ArraySelf4 characterizes the row effect shift upon any gradient that may be observed in array self4, (Row-103.983)*ArraySelf7 characterizes the row effect shift upon any gradient that may be observed in array self7, (Col-215.455)*ArraySelf3 characterizes the column effect shift upon any gradient that may be observed in array self3, (Col-215.455)*ArraySelf4 characterizes the column effect shift upon any gradient that may be observed in array self4, (Col-215.455)*ArraySelf7 characterizes the column effect shift upon any gradient that may be observed in array self7, (Row-103.983)*(Row-103.983)*ArraySelf characterizes the second-order row effect shift (shift/correction relative to the persistent array-independent pattern noted above) upon any gradient that may be observed in array self3, (Row-103.983)*(Row-103.983)*ArraySelf4 characterizes the second-order row effect shift upon any gradient that may be observed in array self4, (Row-103.983)*(Row-103.983)*ArraySelf7 characterizes the second-order row effect shift upon any gradient that may be observed in array self7, (Row-103.983)*(Col-215.455)*ArraySelf3 characterizes the shift/correction relative to the persistent array-independent pattern upon any gradient that may be observed in array self3, (Row-103.983)*(Col-215.455)*ArraySelf4 characterizes the shift/correction relative to the persistent array-independent pattern upon any gradient that may be observed in array self4, (Row-103.983)*(Col-215.455)*ArraySelf7 characterizes the row and column interaction effect shift upon any gradient that may be observed in array self7, (Col-215.455)*(Col-215.455)*ArraySelf3 characterizes the second-order column effect shift upon any gradient that may be observed in array self3, (Col-215.455)*(Col-215.455)*ArraySelf4 characterizes the second-order column effect shift upon any gradient that may be observed in array self4, and (Col-215.455)*(Col-215.455)*ArraySelf7) characterizes the second-order column effect shift upon any gradient that may be observed in array self7.

That is, these metrics provide a measure of array-dependent gradients, i.e., the variation of the gradient pattern from array to array, relative to the persistent, array-independent pattern (estimated as the pattern averaged over all array-specific patterns). Based upon the significance values (<0.05) relative to the parameter sizes, it was determined that the array-dependent gradients are significant, but very small.

Because of the large number of data points (LnRatiOrgDNS values) used in this analysis, a lot of statistical leverage was provided and it was possible to detect very small changes in gradient, much less than a level that was considered significant (i.e., where significance was considered for values of p<0.05).

Therefore, it was concluded that the gradient levels were significant and, if the consequential percent CV levels are above thresholds considered acceptable, then the arrays fail market requirements. The Ln Ratio, array-dependent gradients are also significant, but very small as indicated by the third grouping of parameters and associated statistics.

Table 4 shows the combined statistics for all of the terms described above in Table 3. Rather than reporting p-values for array shifts separately, Table 4 combines the effects over all arrays and provides p-values that were calculated for each term over all arrays. Thus, the information in Table 4 is provided to answer the question as to whether there is an array effect of one ore more terms on the LnRatiOrgDNS data. Table 4 reports ensemble significance, that is the significance of all levels of each term considered together. Terms may also be custom-combined in a manner as taught in co-pending, commonly assigned application Ser. No. 11/198,362.

“Source” lists each of the variables/terms that were considered in performing the ANOVA calculations. DF list the degrees of freedom for the calculations performed for the variable listed in the same row, respectively. For nominal variables, the DF value was the total number of levels (nominal variables) minus one, to account for the intercept, as noted above, and further discussed in application Ser. No. 11/198,362. The Sum of Squares calculations divided by DF, provides the relative weights attributed to the effect of each variable on the LnRatiOrgDNS data. An F-ratio value was calculated for Sum of Squares term and reported in the next adjacent column. From these F-ratio values, p-values were calculated to show the probability that each effect is due to noise, or actually due to the term/variable considered. A p-value of 1 means that there is no evidence at all to suggest that there is a systematic effect caused by the variable/term for which the p-value is calculated. Conversely, a p-value less than 0.0001 means that the result is highly significant, and that the effect (mean sum of squares term, versus the residual mean sum of squares term) calculated for that term is due predominantly to the term considered, and not to random noise. Thus, the lower the p-value, the more significant is the result (i.e., the calculated sum of squares value is more likely to actually be due to the term considered, rather than predominantly to noise). The low Prob>F values in Table 4 imply statistically significant impact, but unacceptable arrays according to typical market requirements, since %CV impact of the effect estimates are small and less than 12%.

TABLE 4
Effect Tests

| Source | DF | Sum of Squares Term | F Ratio | Prob>F |
|---|---|---|---|---|
| Array | 3 | 0.522313 | 12.4062 | <.0001 |
| Row&RS | 1 | 3.633771 | 258.9326 | <.0001 |
| Col&RS | 1 | 0.734277 | 52.3226 | <.0001 |
| Row*Row | 1 | 0.429448 | 30.6013 | <.0001 |
| Row*Col | 1 | 3.625657 | 258.3544 | <.0001 |
| Col*Col | 1 | 16.492148 | 1175.185 | <.0001 |
| Row*Array&RS | 3 | 1.695285 | 40.2671 | <.0001 |
| Col*Array&RS | 3 | 3.863817 | 91.7750 | <.0001 |
| Row*Row*Array | 3 | 0.553207 | 13.1400 | <.0001 |
| Row*Col*Array | 3 | 0.013416 | 0.3187 | 0.8119 |
| Col*Col*Array | 3 | 0.156992 | 3.7289 | 0.0108 |

The total (mean-adjusted) sum of squares calculated was 2049.5670, as indicated in Table 2. The sum of squares calculations for each of the terms considered, as shown in Table 4, are very small relative to the total sum of squares. Thus, although the effects of these terms are statistically significant, as shown by the p-values in the last column of Table 4, the effects are very small compared to the total sum of squares calculation. Thus, the terms considered are not accounting for the large majority of variation in the signal values. Therefore, the overall variation in the signal values analyzed is not due to dye integrity issues. Based on the small gradients as indicated by the magnitudes of the parameters estimates that model the contour plots, as characterized by the results of the ANOVA testing, it was concluded that the signals associated with red dye versus the respective signals associated with green dye were behaving in parallel (i.e., any effect on the signal caused by red dye, if any, was nearly the same as the effect on the signal caused by green dye, if any, across all probes on all arrays, showing inter-array consistency of the dye labels), and that dye integrity was acceptable so as not to effect the reliability of the signal data representing the actual targets binding to probes. Therefore the labeling (red and green dyes) passed the quality test. That is, the dye effect estimates on the signal data were significant, but small and acceptable as to expected consequential impact, as measured by % CV. Statistical significance of the dye effects, by itself, does not imply unacceptable label integrity, but is necessary when the effect estimates exceed a valid threshold value that would imply unacceptable integrity.

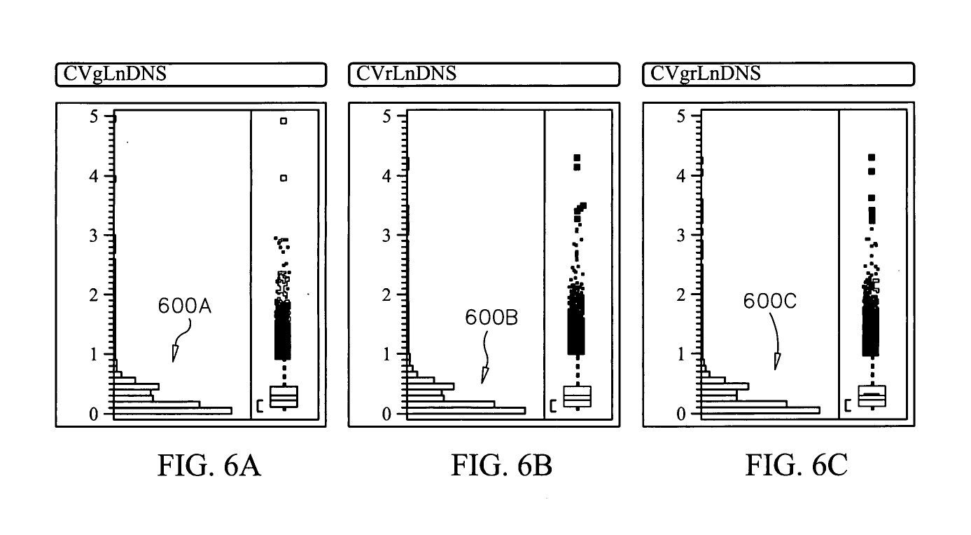

As briefly referred to above, it was determined that the signal intensity readings associated with the different labels may be combined to form a composite or average signal intensity level for a probe, which may be more accurate, reliable and reproducible across experiments than if any single signal intensity level associated with any single label associated with the experiment were used. FIGS. 6A-6C show plots of inter-array coefficient of variation (CV) values (relative noise) 600A, 600B and 600C, respectively plotted for the signals associated with the green dye (Cy3) (FIG. 6A), the signals associated with the red dye (Cy5) (FIG. 6B) and average signals computed from an average of both the signal (FIG. 6C) associated with the red dye and the signal associated with the green dye from each probe (CVgLnDNS, CVrLnDNS and CVgrLnDNS, respectively). In each case the signals were the dye normalized, background-subtracted signals described with regard to the example above for which ANOVA analysis was performed.

Table 5 reports the numerical quantile statistics and moments calculated from the data shown in FIGS. 6A-6C. N represents the total number of data points (number of probes over two different targets) analyzed in each instance.

TABLE 5

| Quantiles-FIG. 6A | | Quantiles-FIG. 6B | | Quantiles-FIG. 6C | |
|---|---|---|---|---|---|
| 100.0% max | 4.9136 | 100.0% max | 4.2909 | 100% max | 4.2824 |
| 99.5% | 1.3502 | 99.5% | 1.4050 | 99.5% | 1.3743 |
| 97.5% | 0.8980 | 97.5% | 0.9610 | 97.5% | 0.9311 |
| 90.0% | 0.5269 | 90.0% | 0.5742 | 90.0% | 0.5443 |
| 75.0% qtle | 0.3977 | 75.0% qtle | 0.4270 | 75.0% qtle | 0.4132 |
| 50.0% med | 0.1719 | 50.0% med | 0.1792 | 50.0% med | 0.1733 |
| 25.0% qtle | 0.0789 | 25.0% qtle | 0.0828 | 25.0% qtle | 0.0800 |
| 10.0% | 0.0314 | 10.0% | 0.0344 | 10.0% | 0.0328 |
| 2.5% | 0.0078 | 2.5% | 0.0088 | 2.5% | 0.0082 |
| 0.5% | 0.0015 | 0.5% | 0.0016 | 0.5% | 0.0017 |
| 0.0% min | 5.59e−6 | 0.0% min | 0.00001 | 0.0% min | 3.12e−6 |
| Moments-FIG. 6A | | Moments-FIG. 6B | | Moments-FIG. 6C | |
| Mean | 0.2562217 | Mean | 0.2742067 | Mean | 0.2640669 |
| Std. Dev. | 0.2448092 | Std. Dev. | 0.2622933 | Std. Dev. | 0.2533214 |
| Std. Err.Mean | 0.0009133 | Std. Err. Mean | 0.0009784 | Std.Err.Mean | 0.0009448 |
| Uppr95%Mean | 0.2580117 | Uppr95%Mean | 0.2761242 | Uppr95%Mean | 0.2659187 |
| Lwr95%Mean | 0.2544317 | Lwr95%Mean | 0.2722891 | Lwr95%Mean | 0.2622151 |
| N | 71856 | N | 71876 | N | 71892 |

The median CV values (array-to-array variability in signal) for Cy3 and Cy5 are 0.1719 and 0.1792, respectively, or 17.19% and 17.92%, which are considered to be unacceptable levels. For example, a typical threshold % CV value considered to be acceptable currently is about 12% or less, sometimes 10% or less. The median CV for the combined signal (FIG. 6C) is 0.1733 or 17.33%, which indicates that the interarray coefficient of variation for the combined signals is as good as for the individual signals, in terms of population statistics. However, the CV for the combined signal is also considered to be unacceptable, as being too high.

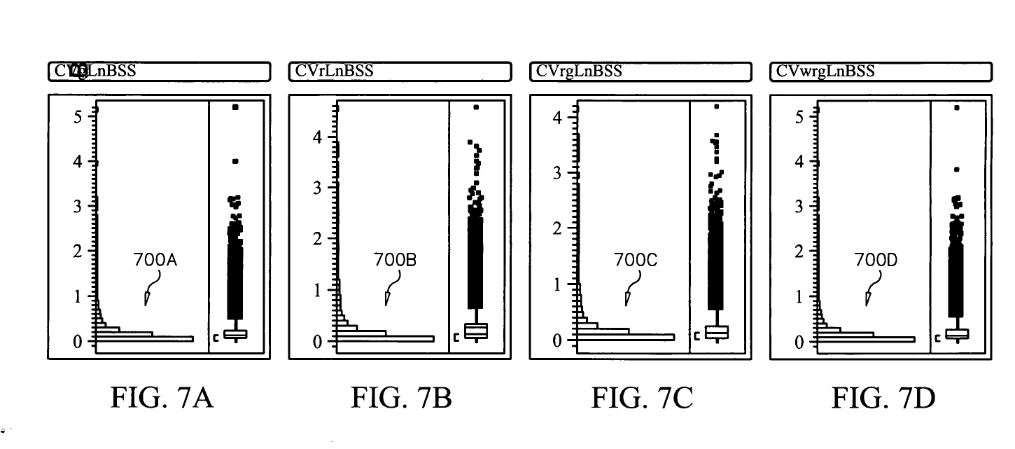

FIGS. 7A-7C show plots of inter-array coefficient of variation (CV) values (relative noise) 700A, 700B and 700C, respectively (CVgLnBSS, CVrLnBSS and CVrgLnBSS, respectively), corresponding to the plots of FIGS. 6A-6C, except in this case, the signals analyzed were not dye-normalized, although they were background-subtracted in the same manner as the signals that are the subject matter of FIGS. 6A-6C. Table 6 reports the numerical quantile statistics and moments calculated from the data shown in FIGS. 7A-7C. N represents the total number of data points analyzed in each instance.

The median CV values (array-to-array variability in signal) for Cy3 and Cy5 are 0.1166 and 0.1204, respectively, or 11.66% and 12.04%, in this case. The median CV for the combined signal (CVrgLnBSS in FIG. 7C) is 0.1143 or 11.43%, which indicates that the interarray coefficient of variation for the combined signals is event better than for the individual signals for the signals that have not been dye-normalized. The reason for the better performance may be that if one of the dyes, for example, performs better at relatively lower signal levels, and the other dye is relatively better performing at relatively higher signal levels, then by averaging both dye related signals at all levels of the spectrum, the impact of the poorer performing dye gets averaged out somewhat by the better performing dye.

TABLE 6

| Quantiles-FIG. 7A | | Quantiles-FIG. 7B | | Quantiles-FIG. 7C | |
|---|---|---|---|---|---|
| 100.0% max | 5.1631 | 100.0% max | 4.5634 | 100% max | 4.1838 |
| 99.5% | 1.5810 | 99.5% | 1.8231 | 99.5% | 1.6959 |
| 97.5% | 1.1269 | 97.5% | 1.3813 | 97.5% | 1.2556 |
| 90.0% | 0.5545 | 90.0% | 0.7870 | 90.0% | 0.5772 |
| 75.0% qtle | 0.2331 | 75.0% qtle | 0.2938 | 75.0% qtle | 0.2537 |
| 50.0% med | 0.1166 | 50.0% med | 0.1204 | 50.0% med | 0.1143 |
| 25.0% qtle | 0.0530 | 25.0% qtle | 0.0521 | 25.0% qtle | 0.0510 |
| 10.0% | 0.0210 | 10.0% | 0.0202 | 10.0% | 0.0199 |
| 2.5% | 0.0052 | 2.5% | 0.0049 | 2.5% | 0.0048 |
| 0.5% | 0.00098 | 0.5% | 0.00099 | 0.5% | 0.00092 |
| 0.0% min | 0.0000 | 0.0% min | 0.00001 | 0.0% min | 0.0000 |
| Moments-FIG. 6A | | Moments-FIG. 6B | | Moments-FIG. 6C | |
| Mean | 0.2154316 | Mean | 0.2660332 | Mean | 0.2369707 |
| Std. Dev. | 0.288496 | Std. Dev. | 0.3651846 | Std. Dev. | 0.3259648 |
| Std. Err.Mean | 0.0010762 | Std. Err. Mean | 0.0013621 | Std.Err.Mean | 0.0012157 |
| Uppr95%Mean | 0.217541 | Uppr9S%Mean | 0.2687029 | Uppr95%Mean | 0.2393535 |
| Lwr9S%Mean | 0.2133221 | Lwr9S%Mean | 0.2633634 | Lwr95%Mean | 0.2345879 |
| N | 71856 | N | 71876 | N | 71892 |

The background-subtracted, but not dye-normalized signals were weighted according to their performances at different relative signal intensities. From experience, it was known that the green dye (Cy3) performs with better integrity (i.e., better reproducibility, less variation, relative to that observed in signals associated with the red dye Cy5) with signals of relatively lower intensity and that the red dye (Cy5) performs with better integrity (i.e., better reproducibility, less variation, relative to that observed in signals associated with the green dye Cy3) with signals of relatively higher intensity. Accordingly, for signals higher than the average signal, rather than just calculating the Ln average of the signal associated with the red dye and the signal associated with the green dye for a probe, the signal associated with the red dye was weighted more heavily than the signal associated with the green dye. Conversely, for signal intensities less than the average signal intensity, the signal associated with the green dye for a probe was weighted more heavily that the signal associated with the red dye for the same probe, and then a log average of these signals was calculated. Thus, signals associated with green dye and having less than the median signal intensity were weighted at a factor of greater than 0.5 and signal associated with red dye having less than the median signal intensity were weighted at a factor of less than 0.5, wherein the weighting factors for red- and green-associated signals from the same probe sum to a total of one. Weighting was performed conversely for the signals having greater than the median signal intensity. A weighting curve was empirically developed to optimize the weighting values applied.

FIG. 7D shows a plot of inter-array coefficient of variation (CV) values (relative noise) 700D (CVwrgLnBSS), corresponding to the plot of FIG. 7C, except in this case, the signals have been weighted in the manner described above. Table 7 reports the numerical quantile statistics and moments calculated from the data shown in FIG. 7D. N represents the total number of data points analyzed.

TABLE 7

| Quantiles-FIG. 7A | | Moments-FIG. 6A | |
|---|---|---|---|
| 100.0% max | 5.1631 | Mean | 0.2194569 |
| 99.5% | 1.5858 | Std. Dev. | 0.294073 |
| 97.5% | 1.1296 | Std. Err.Mean | 0.001097 |
| 90.0% | 0.5772 | Uppr95%Mean | 0.2216071 |
| 75.0% qtle | 0.2508 | Lwr95%Mean | 0.2173067 |
| 50.0% med | 0.1092 | N | 71856 |
| 25.0% qtle | 0.0487 | | |
| 10.0% | 0.0193 | | |
| 2.5% | 0.0047 | | |
| 0.5% | 0.00087 | | |
| 0.0% min | 0.0000 | | |

Note that the median CV value for CVwrgLnBSS is 0.1092 or 10.92%, which is even better (i.e., exhibits less array-to-array variation) than the combined signals of FIG. 7C (CVrgLnBSS) in which equal weighting was applied to signal associated with red dye and signals associated with green dye.

Accordingly, by providing multiple labels for a single sample to be analyzed on an array by interpreting one channel of signals from the array, this offers a unique ability to verify the integrity of each label in a manner that eliminates other production or hybridization factors that may otherwise be confused with effects caused by lack of label integrity. Further, by combining the signals associated with the multiple labels and a particular probe/target, composite signal can be used for measurement of the target. Such composite signal may be more reliable and reproducible than a signal that is associated with any one of the multiple different labels applied to the same sample. Further, weighting may be performed to further emphasize the advantages in the performances of the labels, based on signal intensity.

If unacceptable divergence is identified among the labels, than a user may either have to do the experimentation over (redo the experimentation with new arrays, or strip arrays and repeat the processing) or may be able to identify the bad label and use the results associated with one or more labels that have been determined to be reliable.

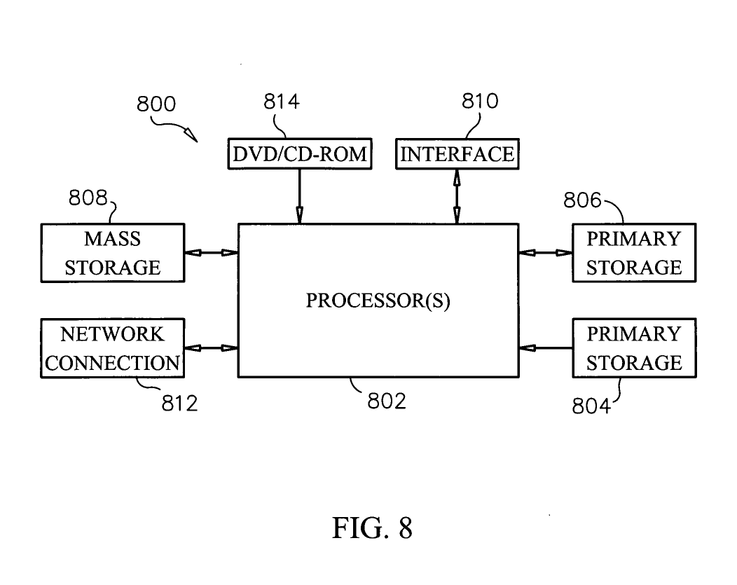

FIG. 8 illustrates a typical computer system in accordance with an embodiment of the present invention. The computer system 800 includes any number of processors 802 (also referred to as central processing units, or CPUs) that are coupled to storage devices including primary storage 806 (typically a random access memory, or RAM), primary storage 804 (typically a read only memory, or ROM). As is well known in the art, primary storage 804 acts to transfer data and instructions uni-directionally to the CPU and primary storage 806 is used typically to transfer data and instructions in a bidirectional manner Both of these primary storage devices may include any suitable computer-readable media such as those described above. A mass storage device 808 is also coupled bi-directionally to CPU 802 and provides additional data storage capacity and may include any of the computer-readable media described above. Mass storage device 808 may be used to store programs, data and the like and is typically a secondary storage medium such as a hard disk that is slower than primary storage. It will be appreciated that the information retained within the mass storage device 808, may, in appropriate cases, be incorporated in standard fashion as part of primary storage 806 as virtual memory. A specific mass storage device such as a CD-ROM or DVD-ROM 814 may also pass data uni-directionally to the CPU. Alternatively, device 814 may be connected for bi-directional data transfer, such as in the case of a CD-RW or DVD-RW, for example.

CPU 802 is also coupled to an interface 810 that may include one or more input/output devices such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 802 optionally may be coupled to a computer or telecommunications network using a network connection as shown generally at 812. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for calculating sums of square terms and or for calculating metrics may be stored on mass storage device 808 or 814 and executed on CPU 808 in conjunction with primary memory 806.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of checking label integrity of labeled biopolymers in a sample assayed by chemical array analysis, said method comprising the steps of:
dividing a sample into equal aliquots;
incorporating at least first and second labels into biopolymers in first and second aliquots of said equal aliquots, respectively;
combining the aliquots to provide a multi-labeled sample comprising sets of identical polymer sequences which are labeled with said at least first and second labels;
hybridizing the multi-labeled sample with probes on a chemical array;
reading signal values from a the probe on the chemical array bound to a set of biopolymer sequences labeled with said at least first and second labels;
comparing first-labeled signal values the probe bound to biopolymer having the first label incorporated therein with second-labeled-signal values from the probe bound to biopolymer having the second label incorporated therein;
repeating said reading signal values and said comparing first-labeled signal values with second-labeled signal values for at least one additional probe on the chemical microarray bound to a set of different biopolymer sequences labeled with said at least first and second labels; and
determining that label integrity is of acceptable quality if divergence between the first-labeled signal values read from the probes and the second-labeled signal values read from the same probes is less than a predetermined threshold value.

2. The method of claim 1, wherein more than two different labels are incorporated into more than two equal aliquots, respectively, and wherein said reading, comparing and determining steps are applied to signals associated with each label in addition to the two labels.

3. The method of claim 1, wherein at least one of the at least two labels is a dye, and wherein the analysis system comprises a scanner.

4. The method of claim 1, wherein said comparing comprises calculating a response surface for each set of signals from each different label incorporated into biopolymers in the sample, relative to the locations of the probes on the array from which the signals were obtained; and comparing contours of the response surfaces to determine the divergence.

5. The method of claim 1, wherein said comparing comprises calculating log ratios of signal pairs, associated with different ones of said at least first and second labels incorporated into biopolymers and bound to the same probe; and calculating differences between the log ratios to determine the divergence.

6. The method of claim 1, further comprising calculating composite signal values from the signal values associated with at least the first and second labels incorporated into biopolymers bound to each probe, when it is determined that label integrity is of acceptable quality.

7. The method of claim 6, wherein said calculating composite signal values comprises calculating average signal values.

8. The method of claim 6, wherein said calculating composite signal values comprises calculating weighted average signal values.

9. A method of checking label integrity of labeled biopolymers hybridized to a chemical array, the labeled biopolymers having been labeled by dividing a sample into equal aliquots, incorporating at least first and second labels into biopolymers in first and second aliquots of said equal aliquots, respectively; combining the aliquots to provide a multi-labeled sample comprising sets of identical polymer sequences which are labeled with said at least first and second labels; and hybridizing the multi-labeled sample with probes on the chemical array; said method comprising the steps of:
reading signal values from a probe on the chemical array bound to a set of biopolymer sequences labeled with said at least first and second labels;
comparing first-labeled signal values the probe bound to biopolymer having the first label incorporated therein with second-labeled-signal values from the probe bound to biopolymer having the second label incorporated therein;
repeating said reading signal values and said comparing first-labeled signal values with second-labeled signal values for at least one additional probe on the chemical microarray bound to a set of different biopolymer sequences labeled with said at least first and second labels; and
determining that label integrity is of acceptable quality if divergence between the first-labeled signal values read from the probes and the second-labeled signal values read from the same probes is less than a predetermined threshold value.

10. A computer readable medium carrying one or more sequences of instructions for checking label integrity of multi-labeled biopolymers in a sample assayed by chemical array analysis, wherein at least first and second labels different from one another have been incorporated into biopolymers in equal aliquots of the sample and then combined to form a multi-labeled sample, and the multi-labeled sample has been hybridized with probes on a chemical array, wherein execution of one or more sequences of instructions by one or more processors causes the one or more processors to perform the steps of:
reading signal values from a probe on the chemical array bound to a set of biopolymer sequences labeled with said at least first and second labels;
comparing first-labeled signal values from the probe bound to biopolymer having the first label incorporated therein with second-labeled signal values from the probe bound to biopolymer having the second label incorporated therein;
repeating said reading signal values and said comparing first-labeled signal values with second-labeled signal values for at least one additional probe on the chemical microarray bound to a set of different biopolymer sequences labeled with said at least first and second labels; and
determining that label integrity is of acceptable quality if divergence between the first-labeled signal values read from the probes and the second-labeled signal values read from the same probes is less than a predetermined threshold value.
